(19) United States
(12) Patent Application Publication
(10) Pub. No.: US 2016/0220367 A1
(43) Pub. Date: Aug. 4, 2016

(54) BALLOON VALVULOPLASTY DELIVERY SYSTEM

(71) Applicant: Medtronic Vascular, Inc., Santa Rosa, CA (US)

(72) Inventor: DONNA BARRETT, Ballybrit (IE)

(21) Appl. No.: 14/613,851

(22) Filed: Feb. 4, 2015

(51) Int. Cl. A61F 2/24; A61M 29/02

(52) U.S. Cl. CPC A61F 2/2427; A61M 29/02

(57) ABSTRACT

A balloon dilation and valve prosthesis delivery device is provided to treat a stenosed valve through balloon dilation and to deliver a valve prosthesis. The device includes an inner shaft assembly including an intermediate portion providing a coupling structure configured to selectively engage a prosthetic valve, and an outer shaft assembly including a delivery sheath capsule, and an expandable balloon attached to a distal end of the outer shaft assembly, and an inflation lumen extending along the length of the outer shaft assembly.

### BACKGROUND OF THE INVENTION

1. Field of the Invention

The present invention is related to a dilation balloon and prosthetic heart valve delivery system.

2. Background Art

Unhealthy Cardiac valves can exhibit two types of pathologies: regurgitation and stenosis. Regurgitation is the more common of the two defects. Either defect can be treated by a surgical repair. In addition, stenosis can be treated through balloon dilation, also known as valvuloplasty, by placing a balloon catheter inside the valve and inflating the balloon in an effort to increase the opening size of the valve and thus improve blood flow.

Under certain conditions, the cardiac valve must be replaced. Standard approaches to valve replacement require cutting open the patient's chest and heart to access the native valve. Such procedures are traumatic to the patient, require a long recovery time, and can result in life threatening complications. Therefore, many patients requiring cardiac valve replacement are deemed to pose too high a risk for open heart surgery due to age, health, or a variety of other factors. These patient risks associated with heart valve replacement are lessened by the emerging techniques for minimally invasive valve repair, but still many of those techniques require arresting the heart and passing the blood through a heart-lung machine.

Efforts have been focused on percutaneous transluminal delivery of replacement cardiac valves to solve the problems presented by traditional open heart surgery and minimally-invasive surgical methods. In such methods, a valve prosthesis is compacted for delivery in a catheter and then advanced, for example, through an opening in the femoral artery and through the descending aorta to the heart, where the prosthesis is then deployed in the aortic valve annulus. Often in the case of a stenosed valve, valvuloplasty is performed prior to delivery of the valve prosthesis. In addition, after deployment of the valve prosthesis, balloon dilation can be performed to post dilate the valve prosthesis and ensure that the valve prosthesis is adequately seated in the native valve annulus.

Balloon valvuloplasty is typically carried out prior to a TAVI (Transcatheter Aortic Valve Implantation) procedure in order to open out the calcified tissue leaflets. Some physicians will also do a second balloon valvuloplasty procedure after the valve has deployed in order to ensure that the valve has fully opened out. This means going in with a balloon catheter, retracting it, going in with the valve delivery system, retracting it, then going back in with the balloon catheter. This adds to procedure time, which adds more potential risk to the patient and can be very laborious for a physician. What is needed is a delivery system that not only gives the physician the choice of using a balloon catheter or to use the valve delivery system, but also allows the physician to select and attach the preferred balloon size for each patient.

### BRIEF SUMMARY OF THE INVENTION

Provided herein is a valve prostheses delivery system that generally includes a delivery system having a capsule at a distal end. The capsule surrounds a compressed valve prosthesis and a balloon is provided on a distal end of the delivery system. Such configurations achieve numerous goals. For example, such a configuration allows for a reduction in the number of devices used to treat a stenosed valve through balloon dilation and to deliver a valve prosthesis. In addition, different types of balloons are interchangeable on the delivery device thereby expanding the treatment options.

In view thereof, disclosed herein are aspects of an balloon dilation and valve prosthesis delivery system which is generally designed to include an inner shaft assembly including an intermediate portion providing a coupling structure configured to selectively engage a prosthetic valve, and an outer shaft assembly including a delivery sheath capsule, and expandable balloon removably coupled to a base tip on a distal end of the outer shaft assembly, and an inflation lumen extending along the length of the outer shaft assembly.

In another exemplary embodiment, disclosed herein are aspects of a balloon dilation and valve prosthesis delivery system including an inner shaft assembly including an intermediate portion providing a coupling structure configured to selectively engage a prosthetic heart valve and an outer shaft assembly including a delivery sheath capsule at a distal end of the outer shaft assembly, the capsule being slidably disposed over the inner shaft assembly and configured to compressively contain a prosthetic heart valve engaged with the coupling structure, an expandable balloon removeably coupled to a distal end of the outer shaft, and an inflation lumen. The inflation lumen extending along the length of the outer shaft assembly, configured to transmit fluid into the balloon for expansion. The inner shaft telescopically slidable within the inflation lumen of the outer shaft assembly, such that a distal end of the inner shaft is extendible forwardly past the distal end of the outer shaft to dispose the inner member within the balloon.

### BRIEF DESCRIPTION OF THE DRAWINGS/FIGURES

The accompanying figures, which are incorporated herein, form part of the specification and illustrate embodiments of a valve prosthesis. Together with the description, the figures further serve to explain the principles of and to enable a person skilled in the relevant art(s) to make, use, and implant the valve prosthesis described herein. In the drawings, like reference numbers indicate identical or functionally similar elements.

FIG. 1 is a sectional view of a valve prosthesis delivery system according to an aspect of this disclosure.

FIG. 2 is a side view of a valve prosthesis delivery system according to an aspect of this disclosure.

FIG. 3 is a perspective view of a valve prosthesis delivery system according to an aspect of this disclosure.

FIG. 4 is a perspective view of a valve prosthesis delivery system with a tip removed according to an aspect of this disclosure.

FIG. 5 is a perspective view of a valve prosthesis delivery system with an inner member extending forwardly according to an aspect of this disclosure.

FIG. 6 is a perspective view of a valve prosthesis delivery system with an inner member extending forwardly according to an aspect of this disclosure.

FIG. 7 is a perspective view of a valve prosthesis delivery system and dilation balloon according to an aspect of this disclosure.

FIG. 8 is a perspective view of a valve prosthesis delivery system and dilation balloon according to an aspect of this disclosure.

FIG. 9 is a perspective view of a valve prosthesis delivery system and dilation balloon according to an aspect of this disclosure.

FIG. 10 is a perspective view of a valve prosthesis delivery system and dilation balloon in a collapsed configuration according to an aspect of this disclosure.

FIG. 11 is a perspective view of a handle to a valve prosthesis delivery system and dilation balloon according to an aspect of this disclosure.

FIG. 12 is a perspective view of a handle to a valve prosthesis delivery system and dilation balloon according to an aspect of this disclosure.

FIG. 13 is a perspective view of a valve prosthesis delivery system with a tip removed and a collet portion drawn in phantom lines to show an inner member disposed within a middle member according to an aspect of this disclosure.

FIG. 14 is a perspective view of a valve prosthesis delivery system with a tip drawn in phantom lines to show a collet portion and an inner member extending forwardly from the collet portion according to an aspect of this disclosure.

FIG. 15 is a schematic view of a stenosed aortic valve and a guide wire for a valve prosthesis delivery system and dilation balloon according to an aspect of this disclosure.

FIG. 16 is a schematic view of a stenosed aortic valve and a valve prosthesis delivery system and dilation balloon according to an aspect of this disclosure.

FIG. 17 is a schematic view of a stenosed aortic valve and a valve prosthesis delivery system and an expanded dilation balloon according to an aspect of this disclosure.

FIG. 18 is a schematic view of a stenosed aortic valve and a valve prosthesis delivery system and a collapsed dilation balloon according to an aspect of this disclosure.

FIG. 19 is a schematic view of a valve prosthesis and valve prosthesis delivery system and dilation balloon according to an aspect of this disclosure.

FIG. 20 is a schematic view of a valve prosthesis and valve prosthesis delivery system and dilation balloon according to an aspect of this disclosure.

FIG. 21 is a schematic view of a valve prosthesis and valve prosthesis delivery system and an expanded dilation balloon according to an aspect of this disclosure.

FIG. 22 is a schematic view of a valve prosthesis after deployment according to an aspect of this disclosure.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician when describing an object or device manipulated by the clinician. “Distal” and “distally” are positions distant from or in a direction away from the clinician. “Proximal” and “proximally” are positions near or in a direction toward the clinician. The terms “distal” and “proximal”, when used with respect to a position in a vessel refer to a position or direction relative to the direction of blood flow. Accordingly, “distal” and “distally” are positions downstream of a reference position, and “proximal” and “proximally” are positions upstream of the reference position.

The following detailed description of a valve prosthesis delivery system refers to the accompanying figures that illustrate exemplary embodiments. Other embodiments are possible. Modifications can be made to the embodiments described herein without departing from the spirit and scope of the present invention. Therefore, the following detailed description is not meant to be limiting. Furthermore, there is no intention to be bound by any expressed or implied theory presented in the preceding technical field, background, brief summary or the following detailed description.

The present invention is directed to a heart valve prosthesis delivery system including a balloon onto a distal portion of the delivery system capsule. The delivery system is a single device that allows a practitioner to perform balloon dilation on native valve leaflets and to deliver a valve prosthesis percutaneously to the heart to replace the function of a native valve. For example, the valve prosthesis can replace a bicuspid or a tricuspid valve such as the aortic, mitral, pulmonary, or tricuspid heart valve.

Typically, balloon dilation, also known as valvuloplasty, is performed using a device separate from the valve prosthesis delivery system. The practitioner first percutaneously inserts the balloon dilation device into the patient, expands the dilation balloon against a native stenosed valve to dilate the valve, deflates the dilation balloon and then removes the balloon dilation device from the patient. At this point, the practitioner can percutaneously insert the valve prosthesis delivery system into the patient to deliver and deploy the valve prosthesis. Occasionally, after the valve prosthesis is deployed, post dilation with the balloon dilation device is required in order to adequately seat the valve prosthesis in the native valve annulus, to prevent valve prosthesis leakage, and/or to remove residual calcification. In this case, the balloon dilation device must be reinserted into the patient after removal of the valve prosthesis delivery system.

An introducer is typically used for a procedure involving balloon dilation and valve prosthesis delivery. The introducer allows for the exchange of the balloon dilation device and valve prosthesis delivery system into and out of the patient. However, the introducer also increases the total size and profile that is inserted into the patient. The profile of a device is the total diameter that must be passed into the patient's vasculature.

Valve prostheses typically have eyelets to attach the valve prostheses to a delivery system. The eyelets attach to tabs which retain the valve prosthesis. In addition, valve prosthesis delivery systems typically include an outer sheath or capsule that surrounds the collapsed valve prosthesis during delivery to the implantation site. During deployment, the capsule is withdrawn over the valve prosthesis.

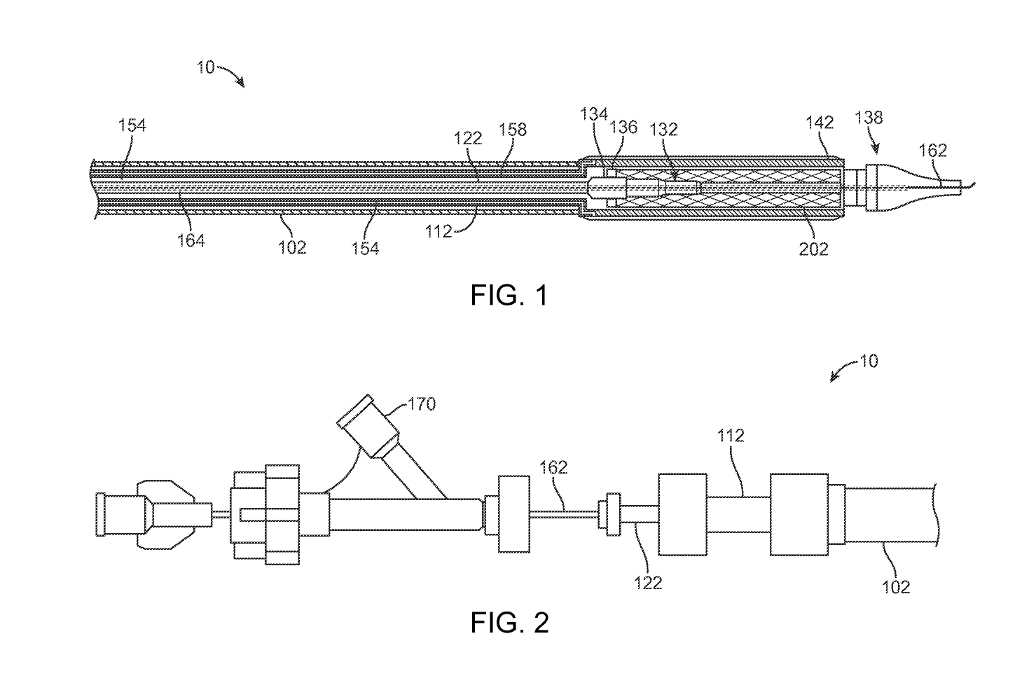

Referring now to FIGS. 1-2, an exemplary delivery system for valve prosthesis 202 includes delivery system 10 that includes an outer sheath 102, a pusher tube or middle member 112, and a central tube or inner member 122, each of which can be concentrically aligned and permit relative motion with respect to each other. At a distal end of middle member 112 is a capsule 142. Middle member 112 also includes an inflation lumen 154. In one aspect of the invention, inflation lumen 154 extends along the length of middle member 112 and is defined by a space between the middle member 112 and a wall 158. In one aspect, inflation lumen 154 is an annular inflation lumen defined by the annular space between wall 158 and middle member 112. In a further aspect, wall 158 is located within middle member 112 such that inflation lumen 154 extends along the interior of middle member 112. In an alternate aspect, wall 158 can surround middle member 112 such that inflation lumen 154 extends along the exterior of middle member 112. In an alternate aspect of the invention, delivery system 10 can include a non-annular inflation lumen and can include one or more single point inflation lumens that extend along the length of middle member 112. The one or more single point inflation lumens can extend along the interior of middle member 112 or along the exterior of middle member 112.

Inner member 122 includes guide wire lumen 164 which passes over guide wire 162. At a distal end of inner member 122 is plunger assembly 132. Capsule 142 surrounds plunger assembly 132 and collapsed valve prosthesis 202 and restrains valve prosthesis 202 in the radial direction during delivery of valve prosthesis 202. In one aspect of the invention, valve prosthesis 202 is self-expandable. In an alternate aspect of the invention, valve prosthesis can be balloon expandable. Plunger assembly 132 includes hub 134 at a proximal end and a tip 138 at a distal end. Tip 138 facilitates the advancement of delivery system 10 through the patient's vasculature. Hub 134 includes one or more tabs 136 for retaining valve prosthesis 202 on plunger assembly 132. Tabs 136 also prevent the pre-release of valve prosthesis 202 and assist in retaining valve prosthesis 202 during recapture. The top surface of tabs 136 interact with the inner surface of capsule 142 to form an interference fit.

Inflation port 170 is connected to inflation lumen 154 and is provided to transmit inflation fluid into balloon 250 to expand balloon 250. Balloon 250 can be manufactured by a person skilled in the art and can utilize common materials including but not limited to Pebax, Grilimid, nylon in various grades, and latex. In one aspect, balloon 250 is a double wall balloon. The double wall thickness of balloon 250 can range from approximately 0.001 inches to approximately 0.005 inches and will be dictated by material and inflation pressure.

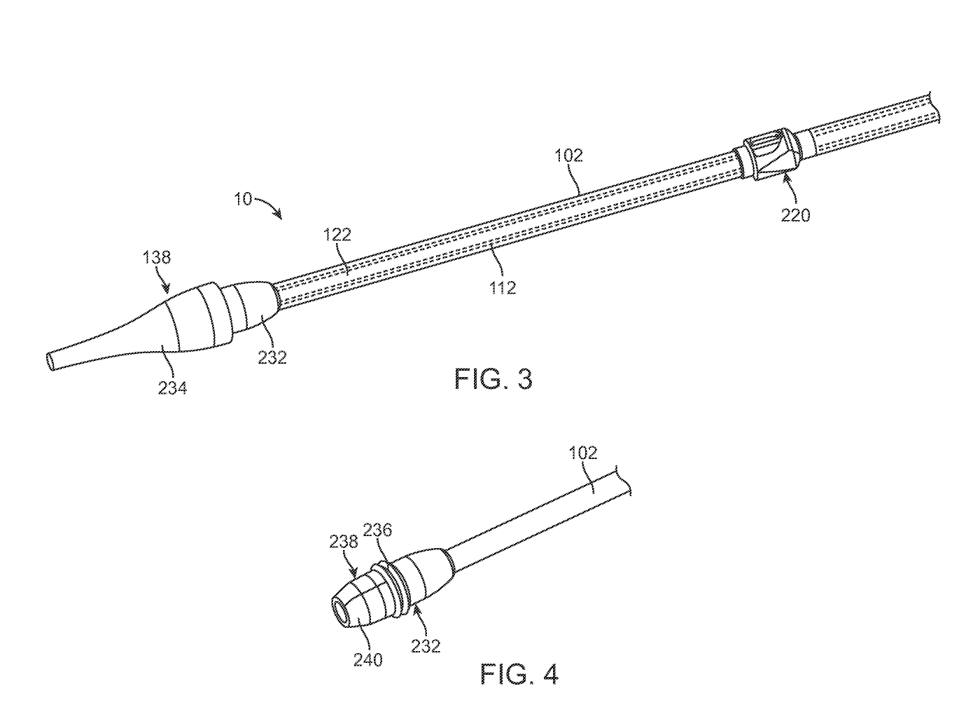

FIG. 3 is an embodiment of delivery system 10 including tip 138 at a distal end thereof, middle member 112, a spindle 220 coupled to middle member 112 and an inner member 122 disposed within middle member 112. Tip 138 includes a base tip 232 adjacent middle member 112 and tip end 234 coupled to base tip 232. Spindle 220 permits inner member 122 to move independently of middle member 112 allowing for telescopic movement of inner member 122 within middle member 112.

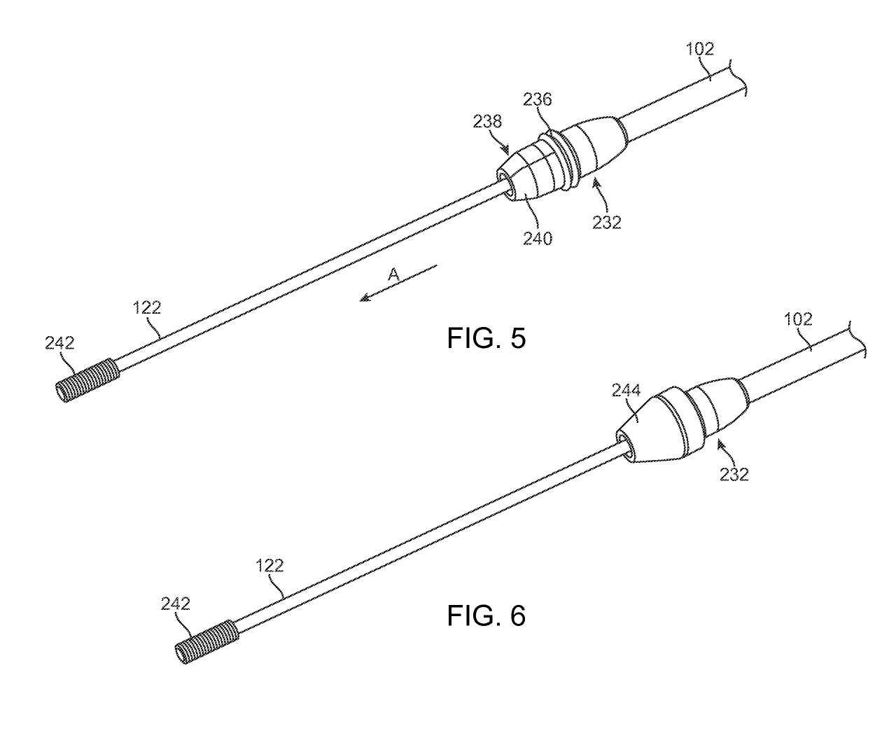

FIG. 4 shows tip end 234 removed from distal end of outer sheath 102. In one embodiment, base tip 232 has threads 236 and has a threaded relationship with tip end 234. In another embodiment, base tip 232 and tip end 234 are removably coupled together by an interference fit. A collet portion 238 extends distally from base tip and includes a plurality of fingers 240 adjacent threads 236. Fingers 240 are deformable in a radial direction when either an axial or radial force is applied to the fingers 240.

FIG. 5 has inner member 122 telescopically extending forwardly (in a direction indicated by arrow A) from a distal end of middle member 112. In one embodiment, inner member 122 has a threaded portion 242 on a distal end thereof. FIG. 6 shows a collet sleeve 244 coupled to base tip 232. In one embodiment, collet sleeve 244 and base tip 232 have a threaded relationship. In another embodiment, collet sleeve 244 and base tip 232 are coupled together by an interference fit (or any other form of locking mechanism that does not have negative impact on profile or outer diameter). As described above, when collet sleeve 244 is coupled to base tip 232, collet sleeve 244 applies axial and radial forces to fingers 240 of collet portion 238, thereby compressing fingers 240 onto inner member 122.

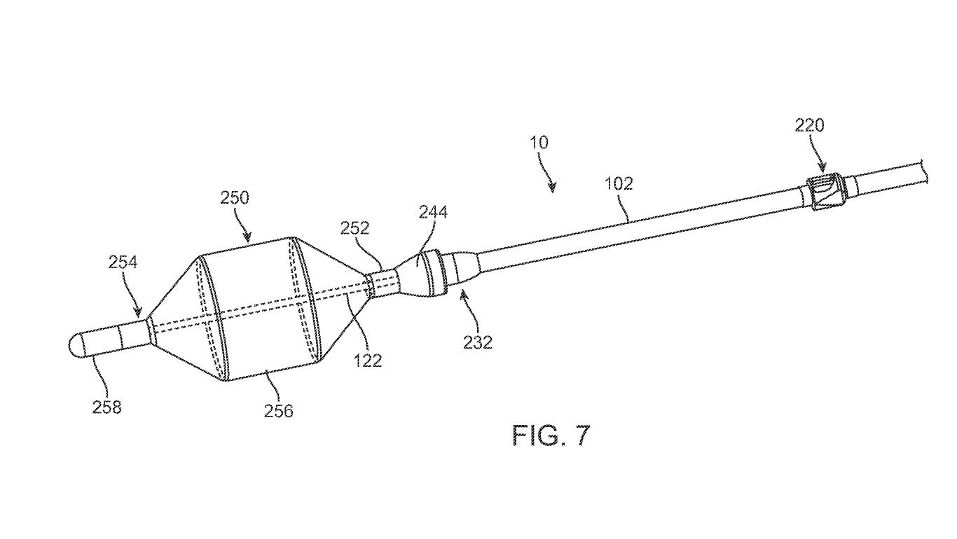

FIG. 7 shows an inflated balloon 250 coupled to the distal end of delivery system 10. Balloon 250 has a first end 252, a second end 254 and a middle inflatable portion 256 disposed therebetween. Inner member 122 telescopically extends forwardly from middle member 112 and disposed within balloon 250. In one embodiment, the unexpanded or wrapped balloon 250 having a first end 252 with an opening which provides access to the interior space of balloon 250 is placed over threaded portion 242 of the forwardly extended inner member 122. Balloon 250 is advanced over inner member 122 until first end 252 is coupled to middle member 112. Notably, a soft cap can be placed at the distal end of inner member 122 to protect the interior of the balloon from the threaded portion 242 as the balloon 250 is advanced over inner member 122.

In the embodiment shown in FIG. 7, threaded portion 242 of inner member 122 passes through an opening in second end 254 and a cap 258 is disposed over second end 254 and is threadedly secured to threaded portion 242 such that second end 254 of balloon 250 is disposed between threaded portion 242 and cap 258 in order to seal second end 254 allowing balloon 250 to be inflated. In one embodiment, cap 258 has a guidewire lumen in fluid communication with guidewire lumen 164 of inner member 122. In another embodiment, cap 258 is a fastener and secured to the exterior surface of threaded portion 242 of inner member 122. Cap 258 is not limited to a threaded relationship with distal end of inner member 122 and other ways to secure cap 258 to inner member 122 are possible, such as an interference fit. In another embodiment, threaded portion 242 of inner member 122 does not pass through second end 254 of balloon 250 and remains disposed within balloon 250. In this embodiment, cap 258 is disposed over threaded portion 242 such that second end 254 of balloon 250 is disposed between threaded portion 242 and cap 258 when cap 258 is securedly coupled to inner member 122.

Integrating balloon onto distal end of shaft allows for the balloon dilation procedure and the valve delivery procedure to be performed using a single device. In addition, post dilation of the valve prosthesis and native valve can be performed with the same delivery device. Because both procedures can be performed with a single device, devices no longer must be exchanged into and out of the body. Therefore, with the delivery system an introducer is no longer necessary thus decreasing the overall device profile that must be inserted into the body to perform the procedures. Reducing the overall profile allows for a smaller insertion hole into the body which leads to a reduction in vessel closure complications. In addition, reducing the number of devices used in the valve repair procedure also decreases the total procedure time. A typical balloon dilation and valve implantation procedure typically requires approximately 20 to approximately 30 minutes of procedure time. Integrating the balloon dilation device into the valve delivery device could save approximately 5 to approximately 10 minutes of total procedure time because a practitioner does not need to exchange a different balloon dilation device and valve prosthesis delivery device. Thus, a patient undergoing the procedure has less time on anesthesia and also has less risk of bleeding. In addition, since balloon 250 is removably coupled to the distal end of delivery system 10, different balloon sizes and types are interchangeable allowing the operator to choose a specific balloon for a procedure. Integrating the balloon dilation device into the valve prosthesis delivery system is beneficial for any access method, including transfemoral, transeptal, transapical, transradial, transsubclavian, or transatrial.

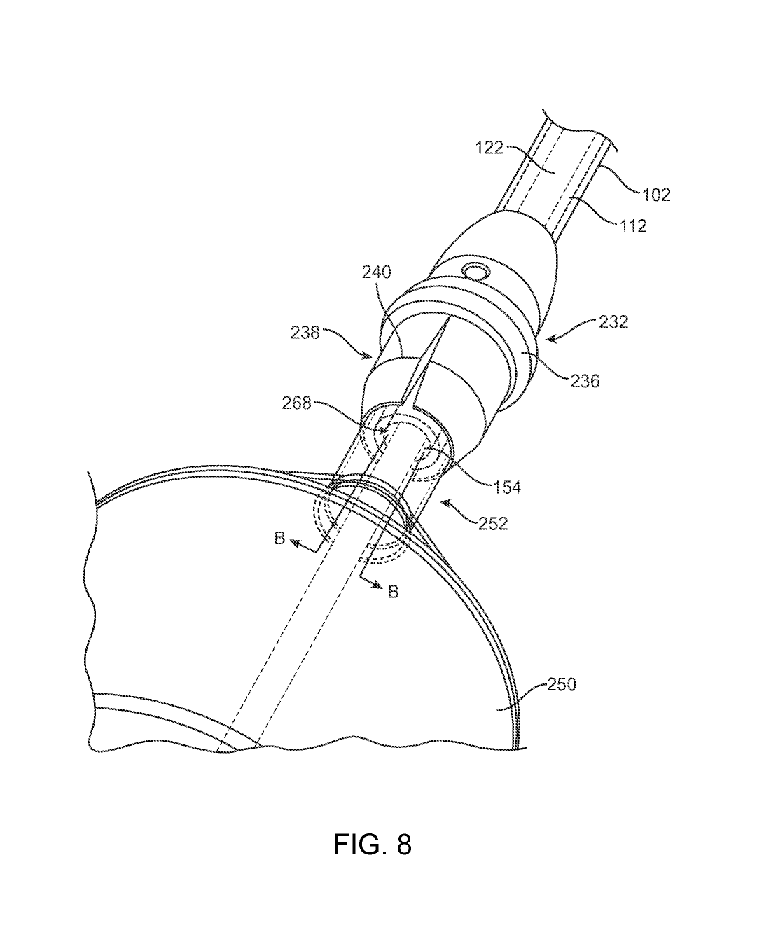

As shown in FIG. 8, collet sleeve 244 is removed to show how a portion of middle member 112 may extend forwardly past base tip 232 such that first end 252 of balloon 250 is disposed between collet portion 238 and middle member 112. In this embodiment, balloon 250 is inflated, airflow travels (as shown by arrow B) through lumen 154 of middle member 112 and into balloon 250.

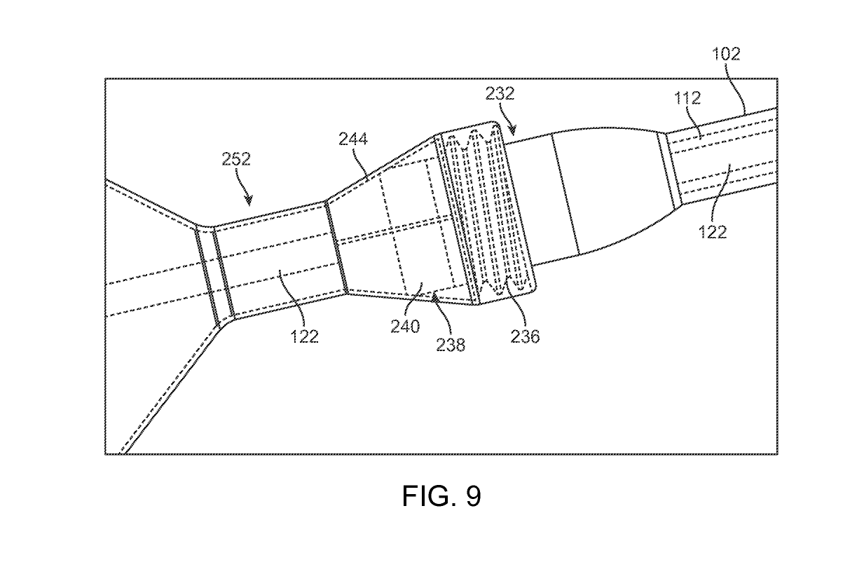

Once first and second ends 252, 254 of unexpanded balloon 250 are secured to middle and inner members 112, 122, respectively, collet sleeve 244 is disposed over balloon 250 and threadedly secured to collet portion 238. Collet sleeve 244 (shown in phantom lines in FIG. 9) secures first end 252 to middle member 112 by compressing fingers 240 of collet portion 238 onto first end 252 of balloon 250. Thus, first end 252 of balloon 250 is disposed between middle member 112 and fingers 240 of collet portion 238. Collet sleeve 244 not only tightens collet portion 238 onto balloon 250, but also reduces leading edges when tracking through a patient's vasculature. In addition, collet sleeve 244 prevents twisting of balloon 250 that might otherwise be happen if collet sleeve 244 is required to be turned in order to secure collet sleeve 244 to collet portion 238. In an optional embodiment, first end 252 of balloon 250 could have a collet sleeve 244 coupled thereto, which avoid the need to preload collet sleeve 244 in a separate step.

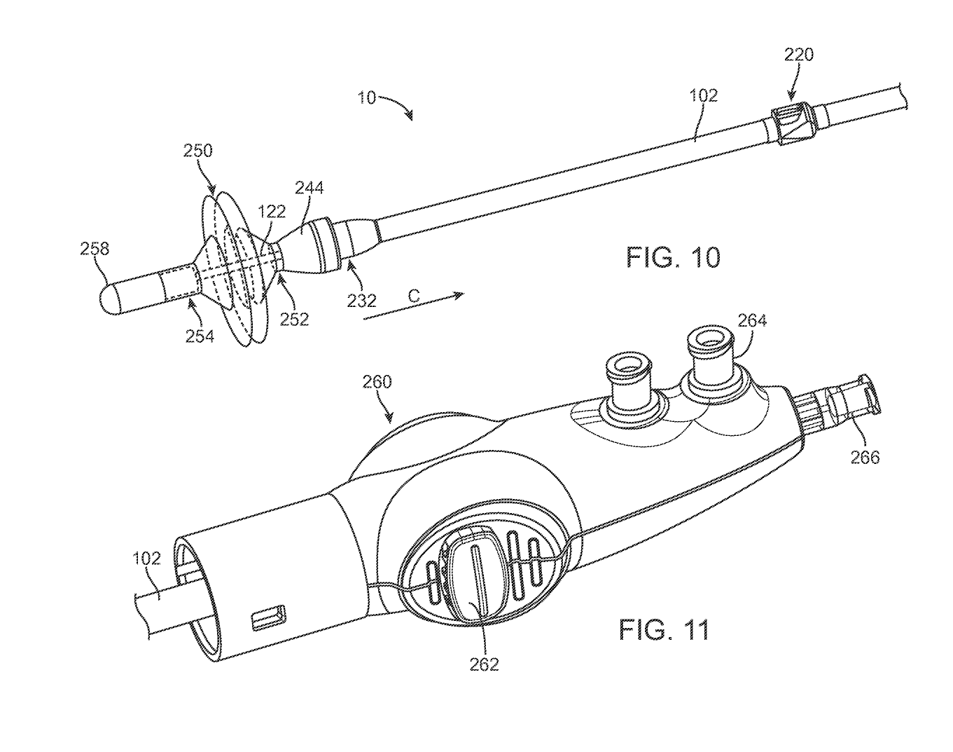

FIG. 10 shows inner member 122 retracting rearwardly within middle member 112 (in the direction of arrow C) thereby collapsing a deflated balloon 250 into a folded configuration. Collapsing balloon 250 into a folded configuration is advantageous because the ventricles have a reduced amount of space so the need to minimize the length of delivery system 10 is critical. In another embodiment, balloon 250 can be collapsed radially onto inner member 122 and inner member 122 does not need to be retracted rearwardly within middle member 112.

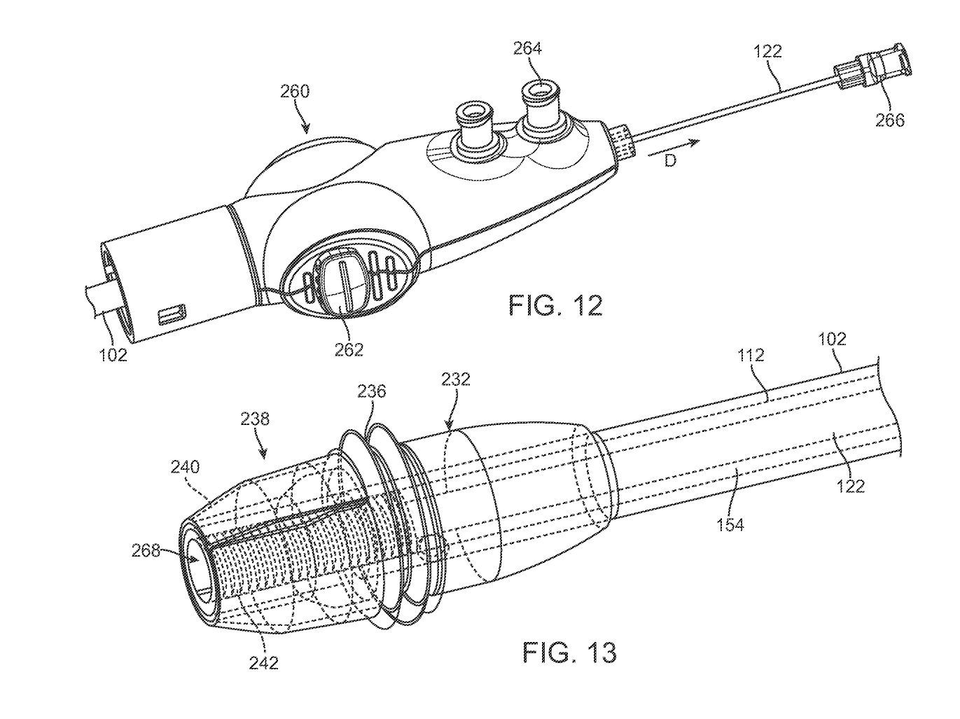

FIG. 11 shows a handle 260 coupled to a proximal end of outer sheath 102. Handle 260 having at least one mechanism 262 for operating delivery system 10 and at least one flushing port 264 for providing a fluid to expand and collapse balloon 250. In one embodiment, inner member 112 has an inner member handle 266 at a proximal end thereof. As shown in FIG. 11, inner member handle 266 is positioned exterior to handle 260 at a proximal end thereof. With the inner member handle 266 disposed on handle 260 this way, a user may grasp inner member handle 266 and telescopic manipulate inner member 122 forwardly and rearwardly within middle member 112. As shown in FIG. 12, inner member 122 is retracted rearwardly within middle member 112 in the direction of arrow D.

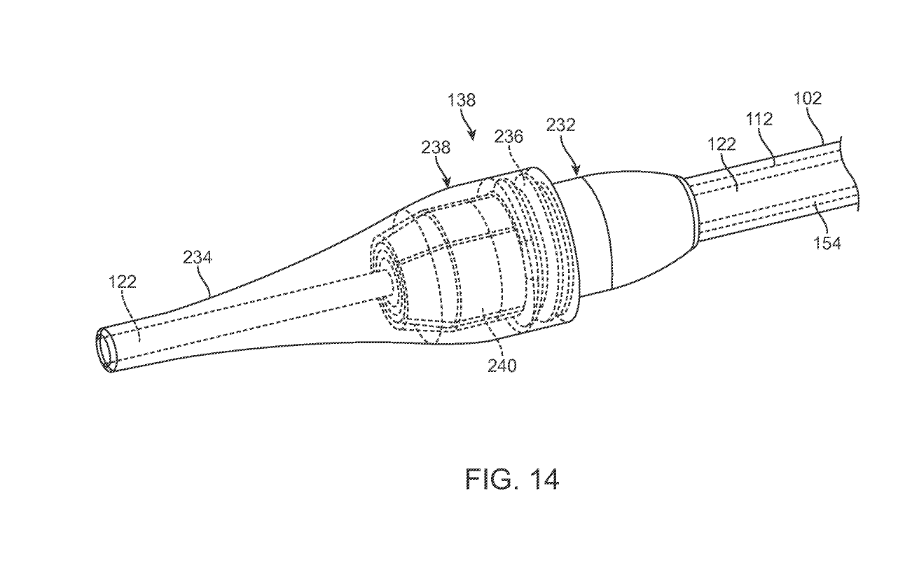

FIG. 13 is a perspective view of distal end of middle member 112 having an opening 268 allowing inner member 122 to telescopically extend forwardly from middle member 112 (as shown in FIG. 5) or retract rearwardly within lumen 154 of middle member 112 (as shown in FIG. 10). Base tip 232 and collet portion 238 are shown in phantom lines to show inner member 122 disposed within middle member 112 such that a distal end of threaded portion 242 is substantially axially aligned with opening 268 of middle member 112. In FIG. 14, tip 138 has tip end 234 shown in phantom lines to exemplify how tip end 234 is secured to base tip 232. In the embodiment shown in FIG. 14, inner member 122 is shown extended forwardly past opening 268 of middle member 112 and further extending within the interior of tip end 234 until inner member 122 abuts distal end of tip end 234.

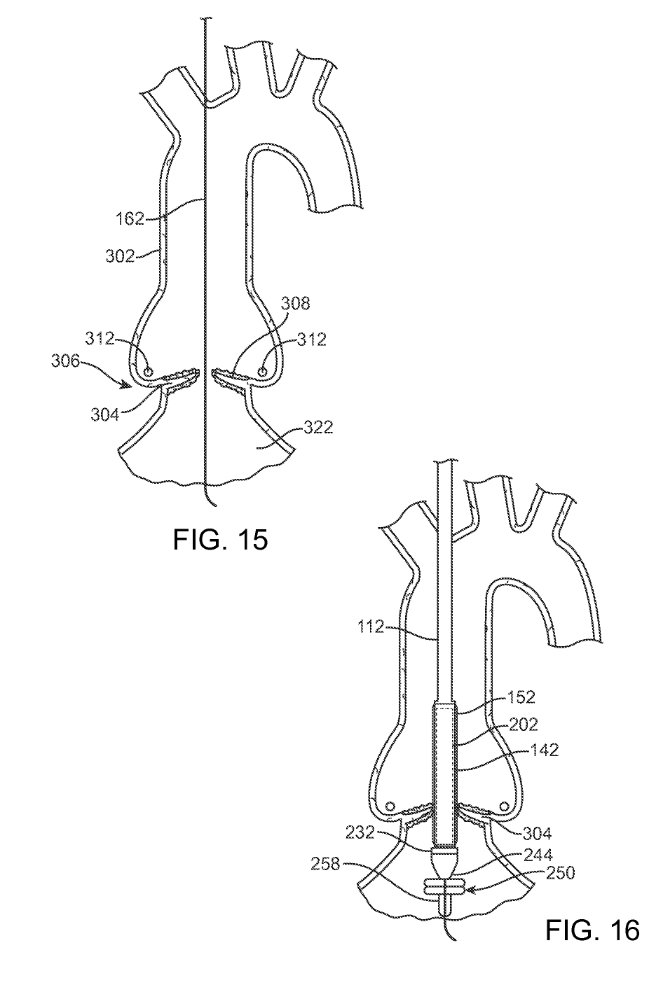

Balloon dilation and implantation of the valve prosthesis will now be described with respect to FIGS. 15-22. As discussed above, in one aspect of the invention the valve prosthesis comprises a self-expanding frame that can be compressed to a contracted delivery configuration onto hub 134 on plunger assembly 132. The self-expanding frame design requires a loading system to crimp valve prosthesis 202 to the delivery size, while allowing the proximal end of valve prosthesis 202 to protrude from the loading system so that the proximal end can be attached to tabs 136.

The valve prosthesis and plunger assembly can then be loaded into capsule 142. In the transfemoral approach, the delivery system and valve prosthesis are advanced into the patient's descending aorta. The delivery system then is advanced, under fluoroscopic guidance, over the aortic arch, through the ascending aorta 302 and into the aortic annulus 306, mid-way across aortic valve 304. In the transsubclavian approach, the delivery system and valve prosthesis are advanced through the subclavian artery into the ascending aorta 302 and into the aortic annulus 306, mid-way across the aortic valve 304.

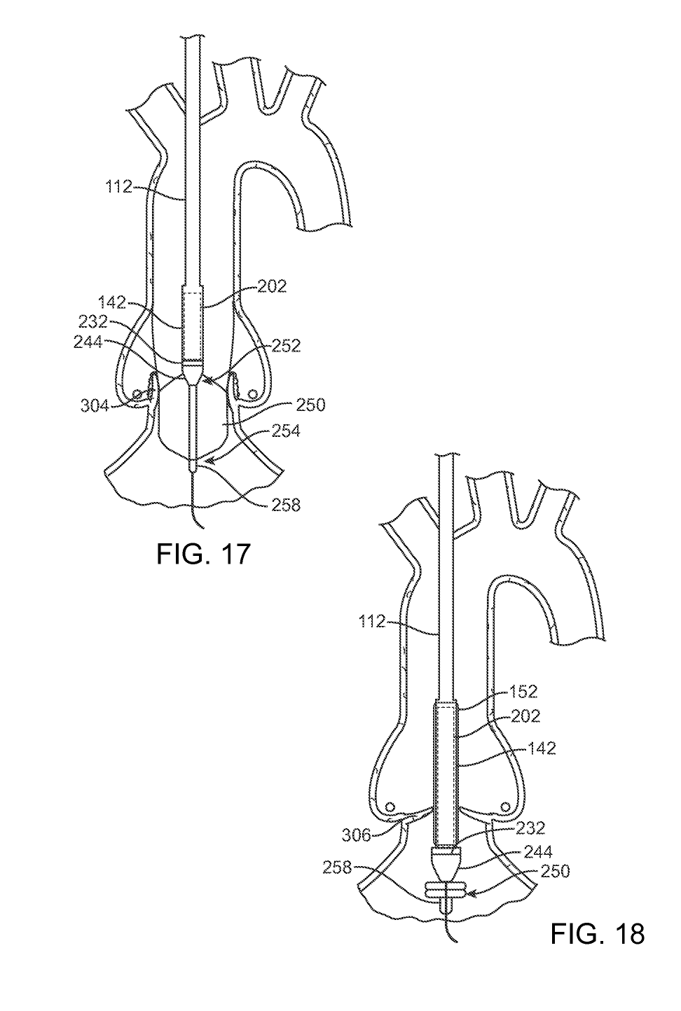

Once positioning of the delivery system in the aortic annulus 306 is confirmed, balloon dilation can be performed by inflating balloon 250 into the native valve leaflets to dilate aortic valve 304 and to treat calcium buildup 308 by deforming the valve leaflets against the aortic wall adjacent aortic valve 304, as shown in FIG. 17. Balloon 250 is expanded by passing fluid through inflation lumen 154 into balloon 250. After balloon dilation is performed, the fluid is removed deflating balloon 250 and inner member 122 is retracted within middle member 112 axially compressing balloon, as shown in FIG. 18.

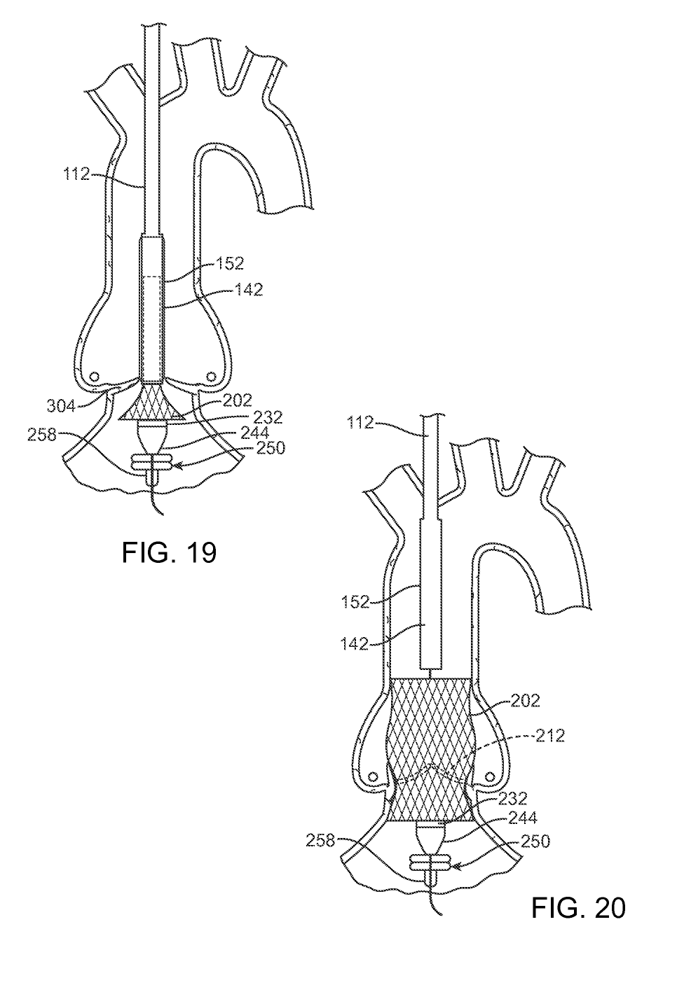

As shown in FIG. 19, after deflation of balloon 152, capsule 142 is withdrawn proximally, thereby permitting valve prosthesis 202 to self-expand. As valve prosthesis 202 expands, it traps the leaflets of the patient's defective aortic valve against the valve annulus, retaining the native valve in a permanently open state. The outflow section of the valve prosthesis expands against and aligns the prosthesis within the ascending aorta, while the inflow section becomes anchored in the aortic annulus of the left ventricle, so that the valve prosthesis skirt reduces the risk of perivalvular leaks, as shown in FIG. 20.

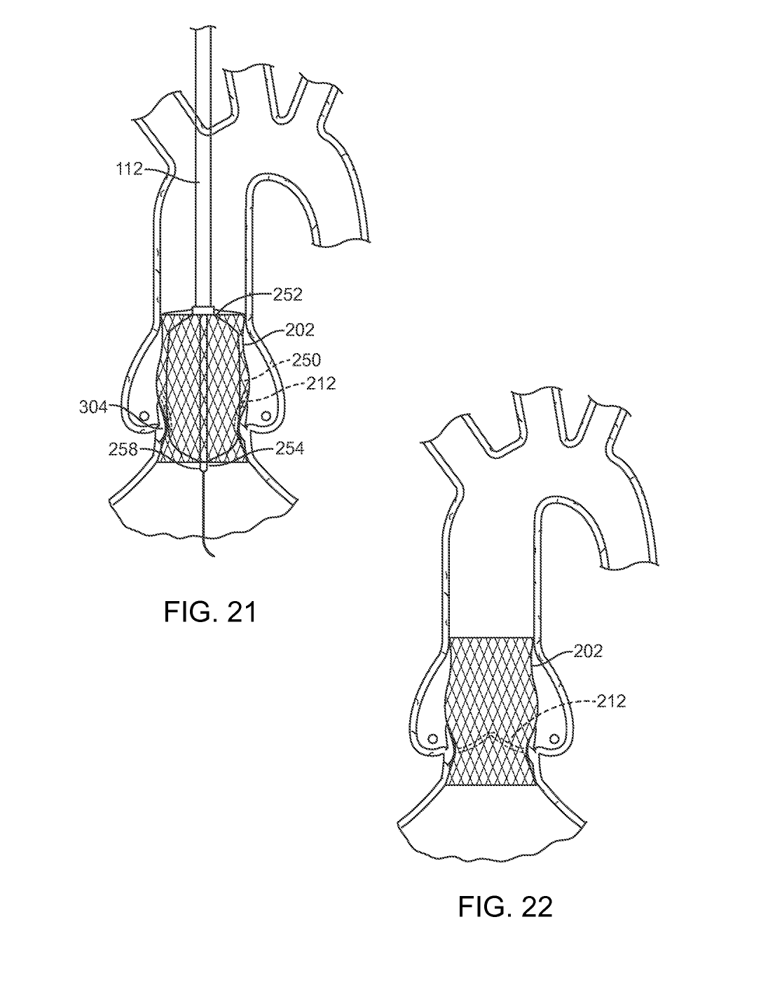

Referring now to FIG. 21, in certain cases, dilation of the prosthetic valve is required after valve delivery in order to properly seat the valve prosthesis, prevent leakage, and/or to remove residual calcification on the native valve. This post valve prosthesis delivery dilation procedure can also be performed using balloon 250 on delivery system 10 after valve prosthesis 202 is delivered and expanded into aortic annulus 306. After deployment of valve prosthesis 202, tip 138 of integrated delivery system 10 is withdrawn proximally to abut the distal end of capsule 142. The integrated delivery system 10 is then advanced into valve prosthesis 202, across replacement valve 212. Once positioning of the delivery system 10 is confirmed, post deployment balloon dilation is performed by inflating balloon 250 into valve prosthesis 202 and aortic annulus 306. FIG. 22 shows the valve prosthesis 202 deployed and expanded into aortic annulus 306 and delivery system 10 is removed from the patient's ascending aorta 302.

Alternatively, the delivery system and valve prosthesis can be advanced through a transapical procedure. In a transapical procedure, a trocar or overtube is inserted into the left ventricle through an incision created in the apex of a patient's heart. A dilator is used to aid in the insertion of the trocar. In this approach, the native valve (e.g. the mitral valve) is approached from the downstream relative to the blood flow. The dilation balloon is attached to an exterior surface of a distal end of the trocar. Balloon dilation is performed by expanding the balloon into the native valve. Then the trocar is retracted sufficiently to release the self-expanding valve prosthesis. The dilator is preferably presented between the valve leaflets. The trocar can be rotated and adjusted as necessary to properly align the valve prosthesis. The dilator is advanced into the left atrium to begin disengaging the proximal section of the valve prosthesis from the dilator. In an alternate aspect of the invention, the delivery system can function as a trocar, thus eliminating the need for an overtube or dilator. In this aspect, tip 138 functions as a trocar to penetrate the incision.

In an alternate aspect of the invention, the valve prosthesis can be delivered through a transatrial procedure. In this procedure, the dilator and trocar are inserted through an incision made in the wall of the left atrium of the heart. The dilator and trocar are advanced through the native valve and into the left ventricle of heart. The dilator is then withdrawn from the trocar. A guide wire is advanced through the trocar to the point where the valve prosthesis comes to the end of the trocar. Balloon dilation is performed by expanding the balloon into the native valve. Then the valve prosthesis is advanced sufficiently to release the self-expanding frame from the trocar. The trocar can be rotated and adjusted as necessary to properly align the valve prosthesis. The trocar is completely withdrawn from the heart such that the valve prosthesis self-expands into position and assumes the function of the native valve. In an alternate aspect of the invention, the delivery system can function as a trocar, thus eliminating the need for an overtube or dilator. In this aspect, tip 138 functions as a trocar to penetrate the incision.

The foregoing description has been presented for purposes of illustration and enablement, and is not intended to be exhaustive or to limit the invention to the precise form disclosed. Other modifications and variations are possible in light of the above teachings. The embodiments and examples were chosen and described in order to best explain the principles of the invention and its practical application and to thereby enable others skilled in the art to best utilize the invention in various embodiments and various modifications as are suited to the particular use contemplated. It is intended that the appended claims be construed to include other alternative embodiments of the invention.

What is claimed is:

1. A balloon dilation and valve prosthesis delivery device comprising:
an inner shaft assembly including an intermediate portion providing a coupling structure configured to selectively engage a prosthetic heart valve; and
an outer shaft assembly including:
a delivery sheath capsule at a distal end of the outer shaft assembly, the capsule being slidably disposed over the inner shaft assembly and configured to compressively contain a prosthetic heart valve engaged with the coupling structure,
an expandable balloon removeably coupled to a distal end of the outer shaft, and
an inflation lumen extending along the length of the outer shaft assembly, configured to transmit fluid into the balloon for expansion.

2. The device of claim 1, wherein the prosthetic heart valve is self-expanding.

3. The device of claim 1, wherein the capsule is configured to compressively contain the prosthetic heart valve within an interior area of the capsule.

4. The device of claim 1, wherein the prosthetic heart valve includes a frame and a plurality of valve leaflets, the frame being configured to engage the coupling structure.

5. The device of claim 1, wherein the balloon has first and second opposing ends and an intermediate section disposed therebetween, at least one of the first and second ends of the balloon removeably coupled to the distal end of the outer shaft assembly.

6. The device of claim 5, wherein at least one of the first and second ends of the balloon is fluidly connected to the inflation lumen.

7. The device of claim 1, wherein the balloon is removeably coupled to a base tip on the distal end of the outer shaft.

8. The device of claim 7, wherein the base tip has a collet portion extending distally therefrom.

9. The device of claim 8, wherein the collet portion includes a plurality of fingers which are deformable in a radial direction when either an axial or radial force is applied to the fingers.

10. The device of claim 8, wherein one end of the balloon is disposed between the outer shaft assembly and the collet portion.

11. The device of claim 10, wherein a collet sleeve is removably coupled to the collet portion to secure one end of the balloon to the base tip.

12. The device of claim 8, wherein a tip end is disposed over collet portion and removeably coupled to the base tip.

13. An balloon dilation and valve prosthesis delivery device comprising:
an inner shaft including a valve prosthesis assembly including an intermediate portion providing a coupling structure configured to selectively engage a prosthetic heart valve; and
an outer shaft assembly including:
a delivery sheath capsule at a distal end of the outer shaft assembly, the capsule being slidably disposed over the inner shaft assembly and configured to compressively contain a prosthetic heart valve engaged with the coupling structure,
an expandable balloon removeably coupled to a distal end of the outer shaft, and
an inflation lumen extending along the length of the outer shaft assembly, configured to transmit fluid into the balloon for expansion, the inner shaft telescopically slidable within the inflation lumen of the outer shaft assembly, such that a distal end of the inner shaft is extendible forwardly past the distal end of the outer shaft to dispose the inner member within the balloon.

14. The device of claim 13, wherein the balloon has first and second opposing ends and an intermediate section disposed therebetween, the first end of the balloon removeably coupled to the distal end of the outer shaft.

15. The device of claim 14, wherein the distal end of the inner member is removeably coupled to the second end of the balloon.

16. The device of claim 15, wherein the distal end of the inner member is secured to the second end of the balloon with a cap.

17. The device of claim 14, wherein the balloon is collapsible in a axial direction when the inner member is retracted rearwardly within outer shaft.

18. The device of claim 13, further comprising a handle coupled to a proximal end of the outer sheath, wherein an inner member handle is disposed exterior to the handle at a proximal end thereof, inner member handle allows for telescopic manipulation of inner member forwardly and rearwardly within outer sheath.
